(19) United States
(12) Patent Application Publication
(10) Pub. No.: US 2020/0282109 A1
(43) Pub. Date: Sep. 10, 2020

(54) Devices and Methods for Repairing Cartilage and Osteochondral Defects

(71) Applicant: New Jersey Institute of Technology, Newark, NJ (US)

(72) Inventors: Treena Lynne Arinzeh, West Orange, NJ (US); George Collins, Maplewood, NJ (US)

(73) Assignee: New Jersey Institute of Technology, Newark, NJ (US)

(21) Appl. No.: 16/813,154

(22) Filed: Mar. 9, 2020

Related U.S. Application Data
(60) Provisional application No. 62/815,780, filed on Mar. 8, 2019.

(51) Int. Cl. A61L 27/36; A61L 27/22; C08L 1/02; A61L 27/20; A61L 27/50; D01D 5/00; D01F 2/24

(52) U.S. Cl. CPC A61L 27/365; A61L 27/222; A61L 27/227; A61L 27/3637; D01F 2/24; C08L 1/02; A61L 27/20; A61L 27/50; D01D 5/0007; A61L 27/3654

(57) ABSTRACT

The present invention provides implants useful for treating cartilage and/or osteochondral defects that comprise a plurality of scaffolds arranged in a multi-layer stacked configuration, wherein each scaffold comprises a mesh of polymer fibers and wherein the polymer fibers comprise gelatin, a plant-derived protein, e.g., zein protein, or a combination thereof. Methods for repairing a cartilage and/or an osteochondral defect using implants of the invention are also provided.

### RELATED APPLICATIONS

This application claims the benefit of priority to U.S. Provisional Patent Application No. 62/815,780, filed on Mar. 8, 2019, the entire contents of which are hereby incorporated herein by reference.

### STATEMENT REGARDING FEDERALLY SPONSORED RESEARCH

This invention was made with government support under Agreement No. DMR 1207173 awarded by the National Science Foundation. The government has certain rights in the invention.

### FIELD OF THE DISCLOSURE

The present disclosure generally relates to biotechnology and regenerative medicine. In particular, the present disclosure is directed to a glycosaminoglycan (“GAG”) mimetic. The present disclosure is also directed to an implant comprising gelatin and/or plant-derived protein scaffolds in a multi-layer stacked configuration.

### BACKGROUND

The general approach to the use of tissue engineering in the repair and/or regeneration of tissue is to combine cells and/or biological factors with a biomaterial that acts as a scaffold for tissue development. The cells should be capable of propagating on the scaffold and acquiring the requisite organization and function to produce a properly functioning tissue.

An estimated 49 million Americans, or 1 of every 6 adults, are affected by cartilage damage, which is projected to increase to 71 million by 2030. Osteoarthritis and related arthritic conditions cost the US $128 billion per year with an estimated $81 billion per year in direct medical costs, and $47 billion per year in indirect costs from loss of wages and productivity. The knee is the most prevalent joint affected, afflicting 16% of the population age 45 and over. Osteoarthritis of the knee is one of the five leading causes of disability amongst non-institutionalized adults, and the third leading cause of number of years lived with disability.

Articular cartilage has a limited intrinsic ability to heal. Clinical intervention is necessary to prevent further cartilage damage and early onset of degenerative osteoarthritis. The natural extracellular matrix (ECM) provides the environment to execute cellular processes responsible for cellular replication, differentiation, maturation, and survival. These processes require profuse cell communication and the biological interplay between cell receptors and protein factors. The presence of fibrocartilage suggests that there is deficient biological activity to promote the chondrocyte phenotype.

GAGs, which are present in native cartilage tissue, provide signaling and structural cues to cells. GAGs are sulfated polysaccharides that are constituent components of the ECM and have been implicated in the stabilizing biological activity of protein factors, as well as facilitating the interaction of protein factors with cell receptors. Specific GAGs, such as chondroitin sulfate and heparin sulfate, are present during cartilage development and their structure may play a role in cartilage formation. GAGs have been shown to interact and maintain the bioactivity of growth factors due to their level and spatial distribution of sulfate groups [1].

Innovative technologies are needed for tissue engineering of inherently complex tissues, and in particular, musculoskeletal connective tissue such as cartilage. Accordingly, compositions and methods that are capable of inducing bone and/or cartilage growth and repair are provided herein.

### SUMMARY OF THE INVENTION

Described herein are compositions and methods useful for promoting the growth and/or differentiation and/or repair of a cell and/or tissue. In certain aspects, the present disclosure includes a scaffold supporting and promoting growth, differentiation, and/or regeneration and repair. The scaffold in one embodiment closely mimics the natural extracellular matrix (ECM) of cartilage.

In accordance with embodiments of the present disclosure, exemplary glycosaminoglycan (GAG) mimetics are used as scaffolds. In some embodiments, exemplary GAG mimetics, derived from cellulose, are utilized as scaffolds for cartilage and wound repair applications.

In one embodiment, cellulose sulfate is employed as a novel GAG mimetic for cartilage tissue engineering. Cellulose sulfate can be tailored to have varying degree and pattern of sulfation similar to native GAGs, chondroitin sulfate-C (CS-C) and heparin sulfate. Chondroitin sulfate-C is chondroitin-6-sulfate. The position of the sulfate is indicated by the number. Heparin sulfate has a sulfate on the 2^{nd} and 6^{th} carbon of the amino sugar. In one embodiment, the present inventors demonstrated the feasibility of cellulose sulfate combined with gelatin as a biomaterial scaffold for cartilage repair. Unlike emerging technologies that add chondrocytes and/or stem cells or growth factors to a scaffold, exemplary embodiments of the present invention do not include any added biological components. In another embodiment, sodium cellulose sulfate (NaCS) is employed.

The fibers/fibrous structure in the scaffold allow for mechanical interlocking of the host tissue with the scaffold during healing to improve adhesion and integration. It will be understood that mechanical interlocking is a phrase used in biomaterials at interfaces when tissues grow into porous structures. In some embodiments, gelatin or gelatin with partially sulfated cellulose (pSC) or fully sulfated cellulose (fSC) are used. In other embodiments, zein protein or zein protein with pSC or fSC are used.

In exemplary embodiments, stacked layers of scaffolds are employed in osteochondral defects. Some embodiments include a gelatin scaffold in the subchondral bone and fSC or pSC-gelatin scaffolds in the cartilage. The present invention can be combined with bone marrow-stimulating techniques for cartilage lesions.

In some embodiments, the present invention provides an implant for promoting bone and/or cartilage formation, the implant comprising a plurality of scaffolds arranged in a multi-layer stacked configuration; wherein each scaffold comprises a mesh of polymer fibers; and wherein the polymer fibers comprise gelatin, a plant-derived protein or a combination thereof.

In some aspects, the polymer fibers in at least one scaffold further comprise a sulfated polymer. In further aspects, the sulfated polymer is selected from the group consisting of cellulose sulfate, starch sulfate and chitin sulfate. In one aspect, the sulfated polymer is cellulose sulfate, e.g., sodium cellulose sulfate (NaCS). In one aspect, the cellulose sulfate is a fully sulfated cellulose sulfate (fSC), partially sulfated cellulose sulfate (pSC) or a combination thereof.

In some aspects, the polymer fibers are electrospun.

In some aspects, the polymer fibers comprise gelatin.

In some embodiments, the polymer fibers are crosslinked. In further embodiments, the polymer fibers are crosslinked with a crosslinker selected from the group consisting of N-(3-dimethyl aminopropyl)-N′-ethyl carbodiimide with N-hydroxysuccinimide (EDC/NHS), genipen and a combination thereof.

In some aspects, the polymer fibers comprise a plant-derived protein. In further aspects, the plant-derived protein is selected from the group consisting of zein protein and soy protein. In one aspect, the plant-derived protein is zein protein.

In some embodiments, the polymer fibers are crosslinked. In further embodiments, the polymer fibers are crosslinked with an epoxy-based crosslinker. In one aspect, the epoxy-based crosslinker is trimethylolpropane triglycidyl ether (TMPGE).

In some aspects, in at least one scaffold the polymer fibers have an average fiber diameter of between about 100 nm and about 100 μm.

In some aspects, in at least one scaffold the mesh of polymer fibers exhibits interfiber spacing of between about 10 μm and about 200 μm.

In some embodiments, the implant is hydrolytically stable. In a further embodiment, in at least one scaffold, polymer fibers exhibit an increase of between about 20% and about 70% in fiber diameter after incubation in an aqueous solution for 1 day and do not exhibit a further statistically significant increase in fiber diameter after incubation in an aqueous solution for more than 1 day and up to 30 days.

In another further embodiment, in at least one scaffold, the mesh of polymer fibers does not exhibit a significant increase in interfiber spacing after incubation in an aqueous solution for up to 30 days.

In yet another further embodiment, at least one scaffold exhibits an increase in weight of between about 50% and about 250% after incubation in an aqueous solution for 1 day and does not exhibit a further statistically significant increase in weight after incubation in an aqueous solution for more than 1 day and up to 30 days.

In yet another further embodiment, at least one scaffold exhibits an increase of between about 5% and about 100% in thickness after incubation in an aqueous solution for 1 day and does not exhibit a further statistically significant increase in thickness after incubation in an aqueous solution for more than 1 day and up to 30 days.

In some aspects, the present invention provides a method for repairing a cartilage and/or an osteochondral defect in a subject in need thereof, the method comprising disposing in the cartilage and/or the osteochondral defect the implant of the invention.

In some aspects, the present invention also provides a method for repairing a cartilage and/or an osteochondral defect in a subject in need thereof, the method comprising disposing in the cartilage and/or the osteochondral defect a plurality of scaffolds arranged in a multi-layer stacked configuration; wherein each scaffold comprises a mesh of polymer fibers; and wherein the mesh of polymer fibers comprises gelatin, a plant-derived protein or a combination thereof.

In some embodiments, the polymer fibers in at least one scaffold further comprise a sulfated polymer. In further embodiments, the sulfated polymer is selected from the group consisting of cellulose sulfate, starch sulfate and chitin sulfate. In one embodiment, the sulfated polymer is cellulose sulfate. In a further embodiment, the cellulose sulfate is a fully sulfated cellulose sulfate (fSC), partially sulfated cellulose sulfate (pSC) or a combination thereof.

In some aspects, the polymer fibers are electrospun.

In some aspects, the polymer fibers comprise gelatin. In some aspects, the polymer fibers are crosslinked. In further aspects, the polymer fibers are crosslinked with a crosslinker selected from the group consisting of N-(3-dimethyl aminopropyl)-N′-ethyl carbodiimide with N-hydroxysuccinimide (EDC/NHS), genipen and a combination thereof.

In some embodiments, the polymer fibers comprise a plant-derived protein. In further embodiments, the plant-derived protein is selected from the group consisting of zein protein and soy protein. In one embodiment, the plant-derived protein is zein protein.

In some aspects, the polymer fibers are crosslinked. In further aspects, the polymer fibers are crosslinked with an epoxy-based crosslinker. In one aspect, the epoxy-based crosslinker is trimethylolpropane triglycidyl ether (TMPGE).

In some embodiments, in at least one scaffold the polymer fibers have an average fiber diameter of between about 100 nm and about 100 μm.

In some embodiments, in at least one scaffold the mesh of polymer fibers exhibits interfiber spacing of between about 10 μm and about 200 μm.

In some embodiments, the plurality of scaffolds arranged in a multi-layer stacked configuration are adapted to the shape of the cartilage defect and/or the osteochondral defect.

In some aspects, a method provided by the present invention is for repairing a cartilage defect and wherein the method further comprises performing a marrow-stimulating technique on the subject. In further aspects, the marrow-stimulating technique is selected from the group consisting of subchondral drilling, abrasion arthroplasty and microfracturing.

In some embodiments, a method provided by the present invention is for repairing an osteochondral defect and wherein the plurality of scaffolds arranged in a multi-layer stacked configuration comprise: at least one scaffold that does not comprise a sulfated polymer; and at least one scaffold that comprises a sulfated polymer; wherein the plurality of scaffolds arranged in a multi-layer stacked configuration is disposed in the osteochondral defect such that the at least one scaffold that does not comprise a sulfated polymer is disposed in the subchondral bone portion of the osteochondral defect, and the at least one scaffold that comprises a sulfated polymer is disposed in the cartilage portion of the osteochondral defect.

In one aspect, the sulfated polymer is cellulose sulfate, e.g., pSC, fSC or a combination thereof.

In some aspects, the present invention also provides a scaffold for promoting bone and/or cartilage formation, the scaffold comprising a mesh of polymer fibers; wherein the polymer fibers comprise a plant-derived protein and a sulfated polymer.

In some embodiments, the plant-derived protein is zein protein.

In some aspects, the sulfated polymer is cellulose sulfate, e.g., pSC, fSC or a combination thereof.

In some embodiments, the polymer fibers are electrospun. In some aspects, the polymer fibers are crosslinked.

In some aspects, the present invention also provides a scaffold for promoting bone and/or cartilage formation, the scaffold comprising a mesh of polymer fibers; wherein the polymer fibers consist essentially of a plant-derived protein.

In some aspects, the plant-derived protein is zein protein.

In some aspects, the polymer fibers are electrospun. In some aspects, the polymer fibers are crosslinked.

In some embodiments, the present invention also provides a method to treat a cartilage defect, comprising: stacking a plurality of scaffolds containing cellulose sulfate in a defect.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed scaffold and associated systems and methods, reference is made to the accompanying figures, wherein:

FIG. 1 is a bar graph showing the amount of production of GAG per cell on fibrous scaffolds after 14 days in growth media with 10% serum. *Significantly greater than gelatin (p<0.05).

FIG. 2, panel (a) is an image of an osteochondral defect. FIG. 2, panel (b) is an image of an implant press-fit into a defect at the time of surgery.

FIG. 3 is a table showing gross images of harvested tissue and histology of rabbit osteochondral defects at 12 weeks. Black arrows show the edges of the defect. H&E and Safranin O. 4× magnification.

FIG. 4 is a schematic showing stacking of electro spun, fibrous crosslinked gelatin or GAG mimetic-containing gelatin for filling osteochondral defects or full-thickness cartilage defects. Each fibrous layer is placed in the defect in a press-fit manner, but loosely packed into the defect allowing for tissue ingrowth. Each layer is a minimum of 0.5 mm thick in this embodiment.

FIG. 5 is a schematic showing an osteochondral defect model using stacked layers of the fibrous crosslinked gelatin in the subchondral bone and the fibrous GAG mimetic-containing gelatin in the cartilage portion of the defect.

FIG. 6, panel (a) is a bar graph illustrating growth of human mesenchymal stem cells (hMSCs) on zein scaffolds compared to gelatin scaffolds, *p<0.05. FIG. 6, panel (b) is a series of confocal images showing hMSCs attachment on gelatin and zein scaffolds with actin staining (green).

FIG. 7, panel (a) is a bar graph showing calcium quantification on zein scaffolds and TCP controls. * indicates statistical significance (p<0.05). FIG. 7, panel (b) is a series of confocal images of MC3T3-E1 cells on zein scaffolds (day 21 in OM conditions) showing osteocalcin (green) and actin filaments (red).

FIG. 8. is a bar graph showing fold change in Hydrated Weight/Original Dry Weight for gelatin and zein scaffolds crosslinked with 10% TMPGE. The higher fold change in hydration is an indicator of less crosslinking. Gelatin has a higher value for all time points as compared to zein.

### DETAILED DESCRIPTION

### Scaffolds of the Invention

In some embodiments the present disclosure provides a scaffold comprising a mesh of polymer fibers, wherein the polymer fibers may comprise, or consist essentially of, a plant-derived protein, e.g., soy protein or zein protein. In other embodiments, the present disclosure also provides a scaffold comprising a mesh of polymer fibers, wherein the polymer fibers may comprise, or consist essentially of, a combination of a plant-derived protein, e.g., soy protein or zein protein, and gelatin.

In some examples, the polymer fibers may further comprise a sulfated polymer, e.g., a sulfated polysaccharide. In some embodiments, the sulfated polysaccharide may be selected from the group consisting of cellulose sulfate, starch sulfate and chitin sulfate. In one specific example, the sulfated polymer is cellulose sulfate, which is a semi-synthetic derivative of cellulose with structural similarity to glycosaminoglycans (GAGs). In some embodiments, cellulose sulfate comprised in the scaffolds described herein may be partially sulfated cellulose (pSC), e.g., cellulose having a sulfate group at the 6^{th} carbon of every alternate glucose unit. In other embodiments, the cellulose sulfate comprised in the scaffolds described herein may be fully sulfated cellulose (fSC), e.g., cellulose having a sulfate group at the 2, 3 and 6^{th} position of every glucose unit. In yet other examples, the cellulose sulfate comprised in the scaffolds described herein may be a combination of pSC and fSC.

In some examples, the polymer fibers comprised in the scaffolds of the present invention may be electro spun.

In some examples, the polymer fibers comprised in the scaffolds of the present invention may be crosslinked. Such polymer fibers may be produced, e.g., by adding a crosslinker to a polymer solution, e.g., a solution comprising a plant-derived polymer, such as zein protein, or a combination of a plant-derived protein and gelatin, prior to electro spinning. Crosslinkers useful in the context of the present invention may be selected from the group consisting of N-(3-dimethyl aminopropyl)-N′-ethyl carbodiimide with N-hydroxysuccinimide (EDC/NHS); genipen, an epoxy-based crosslinker, such as trimethylolpropane triglycidyl ether (TMPGE); and combinations thereof. Specifically, polymer fibers comprising a plant-derived protein, e.g., zein protein, may be crosslinked with an epoxy-based crosslinker, e.g., TMPGE. Polymer fibers comprising gelatin may be crosslinked using EDC/NHS or genipen.

In some examples, the polymer fibers comprised in a scaffold of the present invention may have an average fiber diameter of between about 100 nm and about 100 μM, e.g., between about 100 nm and about 500 nm, between about 100 nm and about 1000 nm, between about 250 nm and about 500 nm, between about 500 nm and about 10 μM, between about 1 μM and about 20 μM, between about 10 μM and about 50 μM, or between about 25 μM and about 100 μM.

In some examples, the mesh of polymer fibers comprised in a scaffold of the present invention may exhibit interfiber spacing of between about 10 μm and about 200 μm, e.g., between about 10 μm and about 50 μm, between about 25 μm and about 75 μm, between about 50 μm and about 150 μm, or between about 100 μm and about 200 μm.

In some examples, a scaffold of the present invention may be hydrolytically stable. The term “hydrolytically stable”, when used to describe a scaffold of the present invention, refers to a scaffold that exhibits certain characteristics when placed in an aqueous solution. For example, the term “hydrolytically stable”, when used to describe a scaffold of the present invention, may refer to a scaffold in which the polymer fibers exhibit an increase of between about 20% and about 70% in fiber diameter after incubation in an aqueous solution for 1 day and do not exhibit a further statistically significant increase in fiber diameter after incubation in an aqueous solution for more than 1 day and up to 30 days.

In some examples, the term “hydrolytically stable”, when used to describe a scaffold of the present invention, may also refer to a scaffold comprising a mesh of polymer fibers that does not exhibit a significant increase in interfiber spacing after incubation in an aqueous solution for up to 30 days.

In some examples, the term “hydrolytically stable”, when used to describe a scaffold of the present invention, may also refer to a scaffold that exhibits an increase of between about 50% and about 250% in weight after incubation in an aqueous solution for 1 day and does not exhibit a further statistically significant increase in weight after incubation in an aqueous solution for more than 1 day and up to 30 days. In some aspects, a scaffold may exhibit an increase of between about 25% to about 75%, or about 50% after incubation in an aqueous solution for up to 21 days.

In some examples, the term “hydrolytically stable”, when used to describe a scaffold of the present invention, may also refer to a scaffold that exhibits an increase of between about 5% and about 100% in thickness after incubation in an aqueous solution for 1 day and does not exhibit a further statistically significant increase in thickness after incubation in an aqueous solution for more than 1 day and up to 30 days.

In some examples, a hydrolytically stable scaffold of the present invention comprises, or consists essentially of, a plant-derived protein, e.g., zein protein.

A hydrolytically stable scaffold is advantageous because it may persist in an aqueous environment, e.g., an aqueous environment of a cartilage defect or an osteochondral defect, for a period of time sufficient to facilitate repair of the cartilage defect or an osteochondral defect.

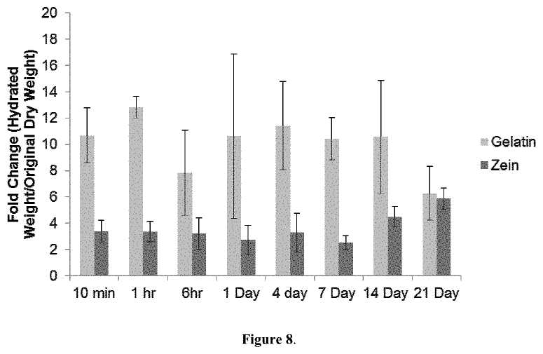

In some examples, a scaffold comprising a mesh of polymer fibers, wherein the polymer fibers consist essentially of zein protein is more hydrolytically stable than a scaffold comprising a mesh of polymer fibers, wherein the polymer fibers consist essentially of gelatin. For example, as illustrated in FIG. 8, a gelatin scaffold crosslinked using 10% TMPGE and soaked in an aqueous solution for up to 21 days displays a higher fold change in weight than a zein protein scaffold crosslinked using 10% TMPGE.

### Implants of the Invention

The present invention also provides implants for promoting bone and/or cartilage formation. The implants of the invention may comprise a plurality of scaffolds arranged in a multi-layer stacked configuration; wherein each scaffold comprises a mesh of polymer fibers; and wherein the polymer fibers comprise, or consist essentially of, gelatin, a plant-derived protein or a combination thereof. In some examples, a plurality of scaffolds may be stacked together inside a cartilage defect and/or an osteochondral defect, thereby producing an implant of the invention directly inside the cartilage defect and/or an osteochondral defect. In other examples, a plurality of scaffolds may be stacked together to produce an implant of the invention prior to disposing the implant inside a cartilage defect and/or an osteochondral defect.

The term “a plurality of scaffolds arranged in a multi-layer stacked configuration”, as used herein, refers to an implant that comprises at least two scaffolds comprising a mesh of polymer fibers that are disposed one on top of another. In some examples, one scaffold may be disposed directly above another scaffold, such that the top scaffold substantially covers the bottom scaffold. In other examples, one scaffold may be offset in relation to another scaffold, such that the top scaffold may only partially cover the bottom scaffold.

In some embodiments, an implant of the present invention may comprise at least two scaffolds arranged in a multi-layer stacked configuration, e.g., 2, 3, 4, 5, 6, 7, 8, 9, 10, 11, 12, 13, 14, 15 or more scaffolds. In some embodiments, the implant may comprise a sufficient number of scaffolds to fill a cartilage defect or an osteochondral defect. In some embodiments, scaffolds of the present invention may be loosely stacked one on top of another to allow space for tissue ingrowth.

In some examples, each scaffold layer has a thickness of at least about 0.5 mm, e.g., about 0.5 mm to about 2 mm, about 1 mm to about 5 mm or about 5 mm to about 10 mm.

The implants of the present invention are useful for promoting bone and/or cartilage formation and for repairing a cartilage and/or an osteochondral defect. In some embodiments, the implants of the present invention may be disposed in a cartilage defect or an osteochondral defect. The stacked configuration of scaffolds in an implant of the invention is advantageous because it allows to adapt the shape the implant to the shape of the cartilage defect or the osteochondrial defect, which, in turn, facilitates healing of the cartilage defect or the osteochondral defect.

The stacked configuration of scaffolds in an implant of the invention is also advantageous because such implant comprises a sufficient amount of polymer fibers that can serve as a surface for cell attachment and tissue ingrowth during cartilage and/or bone repair.

The stacked configuration of scaffolds in an implant of the invention is also advantageous because it allows to combine scaffolds comprising different materials in a single implant, thereby rendering the implant well adapted to facilitate repair of a cartilage and/or an osteochondral defect. For example, an implant may comprise scaffolds comprising a sulfated polymer, e.g., cellulose sulfate, in one portion of the implant, and comprise scaffolds that do not comprise a sulfated polymer in another portion of the implant. Such implant may be disposed in an osteochondral defect such that the portion of the implant comprising the sulfated polymer is disposed in the cartilage portion of the osteochondral defect. In this manner, the sulfated polymer may be placed in the vicinity, or in contact, with the cartilage portion of the osteochondral defect, thereby promoting cartilage repair. At the same time, the non-sulfated portion of the implant may be disposed in the subchondral bone portion of the osteochondral defect, thereby promoting bone repair.

In one example, an implant of the invention may comprise scaffolds comprising, or consisting essentially of, gelatin in the non-sulfated portion, and scaffolds comprising, or consisting essentially of, a combination of gelatin and cellulose sulfate, e.g., fSC and pSC, in the sulfated portion. In another example, the implant comprises scaffolds comprising, or consisting essentially of, zein protein in the non-sulfated portion, and scaffolds comprising, or consisting essentially of, a combination of zein protein and cellulose sulfate, e.g., fSC and pSC, in the sulfated portion.

Implants comprising any number or any combination of various scaffolds described herein are included in the present invention. For example, an implant of the invention may comprise, in any combination, one of more of the following scaffolds arranged in a multi-layer stacked configuration:

a scaffold comprising a mesh of polymer fibers, wherein the polymer fibers comprise, or consist essentially of, gelatin;

a scaffold comprising a mesh of polymer fibers, wherein the polymer fibers comprise, or consist essentially of, gelatin and a sulfated polymer;

a scaffold comprising a mesh of polymer fibers, wherein the polymer fibers comprise, or consist essentially of, a plant-based protein;

a scaffold comprising a mesh of polymer fibers, wherein the polymer fibers comprise, or consist essentially of, a plant-based protein and a sulfated polymer;

a scaffold comprising a mesh of polymer fibers, wherein the polymer fibers comprise, or consist essentially of, a combination of gelatin and a plant-based protein;

a scaffold comprising a mesh of polymer fibers, wherein the polymer fibers comprise, or consist essentially of, a combination of gelatin and a plant-based protein, and a sulfated polymer.

In some examples, the plant-based protein may be selected from the group consisting of soy protein and zein protein. In one specific example, the plant-based protein is zein protein.

The sulfated polymer may be selected from the group consisting of cellulose sulfate, starch sulfate and chitin sulfate. In one specific example, the sulfated polymer is cellulose sulfate, e.g., fSC, pSC or a combination thereof.

In some examples, the plant-derived zein protein may provide advantages over the animal-derived gelatin because of the low immunogenicity of the zein protein.

In some examples, an implant of the present invention may comprise at least one scaffold, wherein the scaffold comprises polymer fibers having an average fiber diameter of between about 100 nm and about 100 μM, e.g., between about 100 nm and about 500 nm, between about 100 nm and about 1000 nm, between about 250 nm and about 500 nm, between about 500 nm and about 10 μM, between about 1 μM and about 20 μM, between about 10 μM and about 50 μM, or between about 25 μM and about 100 μM. In some examples, each scaffold comprised in an implant of the present invention comprises polymer fibers having an average fiber diameter of between about 100 nm and about 100 μM, e.g., between about 100 nm and about 500 nm, between about 100 nm and about 1000 nm, between about 250 nm and about 500 nm, between about 500 nm and about 10 μM, between about 1 μM and about 20 μM, between about 10 μM and about 50 μM, or between about 25 μM and about 100 μM. In some examples, the average fiber diameter is between about 500 nm and about 10 μM, between about 1 μM and about 20 μM, between about 10 μM and about 50 μM, or between about 25 μM and about 100 μM.

In some examples, in implant of the present invention may comprise at least one scaffold, wherein the scaffold comprising a mesh of polymer fibers exhibiting interfiber spacing of between about 10 μm and about 200 μm, e.g., between about 10 μm and about 50 μm, between about 25 μm and about 75 μm, between about 50 μm and about 150 μm, or between about 100 μm and about 200 μm. In some examples, each scaffold comprised in an implant of the present invention comprises a mesh of polymer fibers exhibiting interfiber spacing of between about 10 μm and about 200 μm, e.g., between about 10 μm and about 50 μm, between about 25 μm and about 75 μm, between about 50 μm and about 150 μm, or between about 100 μm and about 200 μm.

Scaffolds comprised in an implant of the present invention may be hydrolytically stable, as described above. For example, an implant of the present invention may comprise at least one scaffold in which the polymer fibers exhibit an increase of between about 20% and about 70% in fiber diameter after incubation in an aqueous solution for 1 day and do not exhibit a further statistically significant increase in fiber diameter after incubation in an aqueous solution for more than 1 day and up to 30 days. In some examples, in each scaffold comprised in an implant of the invention the polymer fibers exhibit an increase of between about 20% and about 70% in fiber diameter after incubation in an aqueous solution for 1 day and do not exhibit a further statistically significant increase in fiber diameter after incubation in an aqueous solution for more than 1 day and up to 30 days.

In some examples, an implant of the present invention may comprise at least one scaffold in which the mesh of polymer fibers does not exhibit a significant increase in interfiber spacing after incubation in an aqueous solution for up to 30 days. In some examples, each scaffold comprised in an implant of the present invention, the mesh of polymer fibers does not exhibit a significant increase in interfiber spacing after incubation in an aqueous solution for up to 30 days.

In some examples, an implant of the present invention may comprise at least one scaffold that exhibits an increase of between about 50% and about 250% in weight after incubation in an aqueous solution for 1 day and does not exhibit a further statistically significant increase in weight after incubation in an aqueous solution for more than 1 day and up to 30 days. In some aspects, the at least one scaffold may exhibit an increase of between about 25% to about 75%, or about 50% after incubation in an aqueous solution for up to 21 days. In some examples, each scaffold comprised in an implant of the present invention exhibits an increase of between about 50% and about 250% in weight after incubation in an aqueous solution for 1 day and does not exhibit a further statistically significant increase in weight after incubation in an aqueous solution for more than 1 day and up to 30 days.

In some examples, an implant of the present invention may comprise at least one scaffold that exhibits an increase of between about 5% and about 100% in thickness after incubation in an aqueous solution for 1 day and does not exhibit a further statistically significant increase in thickness after incubation in an aqueous solution for more than 1 day and up to 30 days. In some examples, each scaffold comprised in an implant of the present invention exhibits an increase of between about 5% and about 100% in thickness after incubation in an aqueous solution for 1 day and does not exhibit a further statistically significant increase in thickness after incubation in an aqueous solution for more than 1 day and up to 30 days.

In some examples, a hydrolytically stable implant of the present invention comprises at least one scaffold, wherein the scaffold comprises, or consists essentially of, a plant-derived protein, e.g., zein protein. In some examples, each scaffold comprised in a hydrolytically stable implant of the present invention comprises, or consists essentially of, a plant-derived protein, e.g., zein protein.

### Methods of the Invention

The present invention also provides methods for repairing a cartilage defect and/or an osteochondral defect that comprise disposing in the cartilage defect and/or an osteochondral defect a scaffold or an implant as described above. For example, a method for repairing a cartilage defect and/or an osteochondral defect may comprise disposing a plurality of scaffolds arranged in a multi-layer stacked configuration in the cartilage defect and/or an osteochondral defect. In some examples, each scaffold layer has a thickness of at least about 0.5 mm, e.g., about 0.5 mm to about 2 mm, about 1 mm to about 5 mm, about 5 mm to about 10 mm, about 5 mm to about 15 mm, about 10 mm to about 25 mm, about 15 mm to about 50 mm or about 35 mm to about 100 mm. In some examples, thickness of an implant disposed in the cartilage defect and/or an osteochondral defect is sufficient to fill the cartilage defect and/or an osteochondral defect. In some examples, thickness of the plurality of scaffolds arranged in a multi-layer stacked configuration is sufficient to fill the cartilage defect and/or an osteochondral defect.

In some examples, a method of the present invention may comprise stacking a plurality of scaffolds as described herein in a cartilage defect and/or an osteochondral defect, thereby creating an implant of the present invention directly inside the cartilage defect and/or an osteochondral defect. For example, a method for repairing an osteochondral defect may comprise stacking a plurality of scaffolds directly inside the osteochondral defect, such that a scaffold that does not comprise a sulfated polymer is disposed in the bone portion of the osteochondral defect and a scaffold that comprises a sulfated polymer is disposed in the cartilage portion of the osteochondral defect.

In other examples, a method of the present invention may comprise disposing a pre-formed implant of the invention in the cartilage defect and/or an osteochondral defect. Any scaffold or an implant described herein may be used in the methods of the present invention.

In some examples, the plurality of scaffolds arranged in a multi-layer stacked configuration may be adapted to the shape of the cartilage defect and/or the osteochondral defect.

In some examples, methods of the present invention may be combined with other methods for stimulating cartilage and/or bone repair. For example, when stimulation of cartilage repair is desired, a method of the present invention may be carried out in combination with a marrow-stimulating technique to promote cartilage repair. In some examples, a marrow-stimulating technique may be selected from the group consisting of subchondral drilling, abrasion arthroplasty and microfracturing.

In some examples, methods of the present invention are not associated with adverse reaction and/or inflammation of the tissues around the cartilage defect and/or the osteochondral defect.

In some examples, methods of the present invention stimulate production of proteoglycan in the cartilage defect and/or the osteochondral defect. In some examples, methods of the present invention stimulate production of collagen in the cartilage defect and/or the osteochondral defect. In some examples, methods of the present invention comprise attachment and/or differentiation of cells, e.g., mesenchymal stem cells, to the polymer fibers comprised in the scaffolds and/or implants of the invention.

The present inventors evaluated cellulose sulfate, having varying degrees of sulfation, in promoting human mesenchymal stem cell (MSC) chondrogenesis in vitro and in vivo in the repair of osteochondral defects.

The materials and the methods of the present disclosure used in some embodiments will be described below. While the embodiments discuss the use of specific compounds and materials, it is understood that the present disclosure could employ other suitable materials. Similar quantities or measurements may be substituted without altering the methods embodied below.

Scaffold Fabrication and In Vitro Cell Study: In this embodiment, 5% (w/w) partially sulfated cellulose (pSC) or fully sulfated cellulose (fSC) was combined with gelatin (bovine gelatin, Sigma) and electrospun to form fibrous scaffolds, using published protocols [2]. pSC has a sulfate group at the 6th carbon of every alternate glucose unit and fSC has a sulfate group at the 2, 3 and 6th position of every glucose unit. Gelatin alone scaffolds were used as a control. MSCs were evaluated for chondrogenesis on the scaffolds by collagen type 2 and GAG production and cell morphology. Cells were grown in growth media without inductive factors to evaluate cellulose sulfate in promoting chondrogenesis.

In another embodiment, 5% (w/w) partially sulfated cellulose (pSC) or fully sulfated cellulose (fSC) was combined with gelatin (bovine gelatin, Sigma) and electrospun to form fibrous scaffolds. Solutions containing 24% (w/w) gelatin from bovine skin type B and either 5% (w/w) pSC or fSC in deionized water (DI water) were prepared in a 60° C. water bath. Also, depending on the ambient humidity and the solubility of the sulfated cellulose, the solvent could include acetic acid.

In this embodiment, the cellulose sulfate was initially measured and mixed in the deionized water until it dissolved, followed by sonication (Branson digital sonifier 450) at 22% amplitude for 2-5 minutes for pSC and 15 minutes for fSC to ensure the uniform distribution of the GAGs through the solvent. Acetic acid was added to the solvent if needed depending on the humidity levels and solubility of the GAG mimics. The solution was mixed in a 60° C. heated water bath on a magnetic stir plate for 10 minutes. Gelatin was added to the solvent and allowed to mix until all of it dissolved. The solution was allowed to remain still in a 60° C. water bath to remove air bubbles.

In this embodiment, electrospinning technique was utilized to create non-woven fibrous scaffolds. The syringe containing the gelatin solution or gelatin with pSC or fSC was maintained constantly at 60° C. using a heating chamber. The solution was charged by applying a high positive voltage charge of 40 kV to a 14-gauge steel needle attached to the syringe containing the solution. A negative voltage charge of 20 kV was applied to a flat stable collector to allow the electrospinning of the gelatin solution on the collector. Approximately 30 cm distance was maintained between the needle and the collector. The flow rate of gelatin solution was maintained at 6.5 ml/hour to stabilize the fiber size and reduce the accumulation of droplets on the collector. Optimum humidity of 25-30% and temperature of 24° C. was maintained in the electrospinning chamber through the process.

Since the gelatin based fibers tend to be unstable in aqueous solutions, they were crosslinked using N-(3-dimethyl aminopropyl)-N′-ethyl carbodiimide (EDC) with N-hydroxysuccinamide (NHS). The crosslinking was used by dissolving the crosslinker in 200 proof ethanol and soaking the electrospun gelatin scaffolds in this solution for 96 hours at room temperature. After incubation with crosslinker, any remaining solution was discarded and the scaffolds were incubated in 0.1M sodium phosphate dibasic (Fisher Scientific) containing solution for two hours to wash off the byproducts of the crosslinker. This was followed by washing the scaffolds in DI water thrice. The scaffolds were allowed to air dry until usage. The EDC-NHS crosslinker can also be substituted with epoxy-based crosslinkers, such as TMPGE (trimethylolpropane triglycidyl ether), where the present inventors have demonstrated that they can successfully crosslink gelatin with TMPGE (up to 10 w/w %) by adding the TMPGE directly to the electrospinning solution prior to electrospinning.

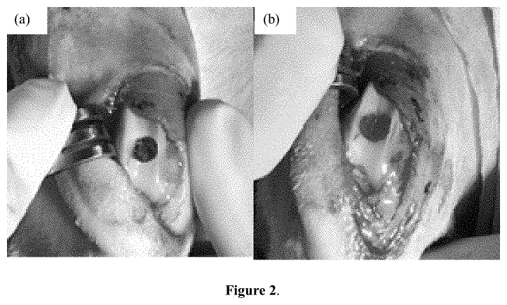

In Vivo Study: Osteochondral defects (5 mm diam.×5 mm. depth) were created bilaterally in the trochlear groove of New Zealand White rabbits (male, skeletally mature 6-7 mos.), as shown in FIG. 2a. Defects were press-fit with biphasic fibrous implants consisting of a top layer of either 5% fSC/gelatin, 5% pSC/gelatin, or gelatin alone, with a thickness of ˜0.5 mm, and a bottom layer of gelatin alone with a thickness of ˜4.5 mm (n=5 per group), as shown in FIG. 2b.

A plurality of scaffolds was stacked in the defect to create the appropriate thicknesses. Each scaffold was at a minimum 0.5 mm thick and press-fit into the defect. It will be understood that the thickness of the scaffold could vary. In the subchondral region, the layers were loosely stacked on top of one another to allow for sufficient space for tissue ingrowth. For the cartilage portion of the defect, only one layer was used since the cartilage defect in this model is 0.5 mm thick. For larger animals and in humans where the cartilage thickness will be >0.5 mm, the scaffolds can also be stacked in a similar manner. In exemplary embodiments, the scaffolds are physically stacked on top of one another, and are not connected to one another chemically.

Defect only group was used as a control (n=4). At 12 weeks, tissues were harvested, fixed in 10% normal buffered formalin, and processed for decalcified paraffin-embedded histology. Sagittal sections were stained with H&E, Safranin O, and Toluidine Blue. Semi-quantitative histological scoring was performed using the International Cartilage Repair Society (ICRS) recommended guidelines for histological endpoints for cartilage repair.

Statistical Analysis: Analysis of variance (ANOVA) was used to determine statistical significance (p<0.05). Tukey's post hoc test was used for statistical differences at p<0.05. All statistics were performed in SPSS Statistics Version 24 (IBM, Armonk, N.Y.).

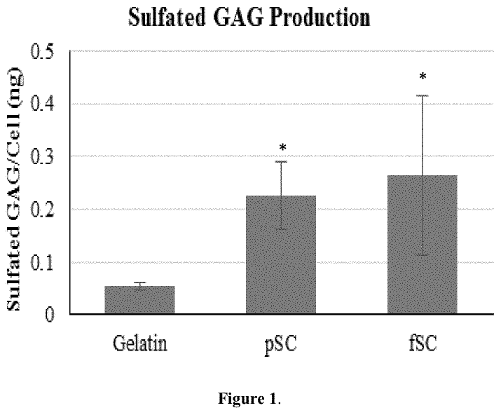

The results of the in vitro and in vivo studies will be described below. In vitro cell study: After 14 days in growth media, cells on pSC and fSC containing scaffolds produced significantly more GAGs than cells on gelatin alone (p<0.05), as shown in FIG. 1 and appeared to produce more collagen type II, as observed by immunostaining.

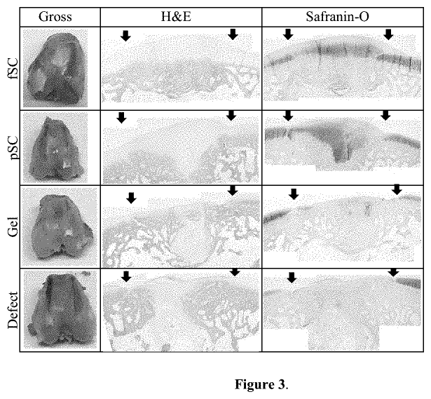

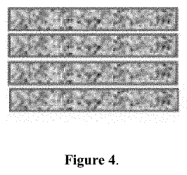

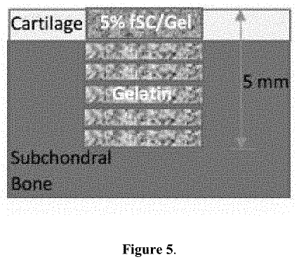

In Vivo Study: At 12 weeks postimplantation, no signs of an adverse reaction/inflammation of the harvested tissue were detected. More cartilage, as shown by the uniform red proteoglycan stain using Safranin-O, was observed for the fSC group as compared to pSC and Gel groups, as shown in FIG. 3. In addition, the fSC group appeared to have more subchondral bone fill than the other implant groups as seen in the H&E stain.

Previous studies have shown that scaffolds containing cellulose sulfate can bind growth factors, such as transforming growth factor-beta3 (TGF-β3), more readily than gelatin alone [2-3]. As is evident from the results, the increased GAG production of cells on scaffolds made with cellulose sulfate suggest specific proteins or growth factors from the serum and/or secreted by the cells could bind to the scaffold, promoting chondrogenesis. fSC group exhibited more uniform cartilage and increased subchondral bone fill, suggesting more advanced healing than the other implant groups. This suggests fSC may be attracting endogenous factors that may facilitate healing.

In some embodiments, the fSC-gelatin scaffolds can also be combine with marrow-stimulating techniques, which are common clinical treatments to treat cartilage lesions. Marrow-stimulating techniques include subchondral drilling, abrasion arthroplasty, and microfracturing. In microfracturing, which is the preferred method and standard-of-care, multiple holes made in the subchondral bone allow cells from the bone marrow to migrate to the joint surface and facilitate repair. Full-thickness chondral defects requiring microfracture, where uniform microfracture holes are made in the subchondral bone, can be treated with the fSC-gelatin scaffold by press-fitting the scaffold into the defect and layering/stacking the scaffolds to fill the thickness of the cartilage. Thus, the scaffolds can be stacked/layered into cartilage lesions in combination with microfracture/marrow-stimulating techniques to repair cartilage defects. Or, in the case of osteochondral defects, gelatin scaffolds are stacked in the subchondral bone portion of the defect with fSC-gelatin or pSC-gelatin scaffolds in the cartilage portion of the defect.

In some embodiments, fiber dimensions can range from 1-10 microns. Interfiber spacing can be a minimum of 15 microns. It will be understood that the fiber dimensions and interfiber spacing could vary. Any suitable crosslinker could be used, such as EDC, EDC-NHS, or epoxy-based crosslinkers. Cellulose sulfate can be combined with other proteins, such as zein, to form fibers.

In exemplary embodiments, a plant derived protein, such as zein, can be a substitute for gelatin. Plant derived proteins are renewable, abundant and have low immunogenic effects. Zein, a protein found in corn, has been studied as a potential scaffold due to its cytocompatibility, antibacterial properties and biodegradability [4, 5]. In the following studies, the present inventors evaluated electrospun zein scaffolds for cell adhesion and growth using human mesenchymal stem cells (hMSCs). These results were compared to gelatin scaffolds, which is denatured collagen and well established for its favorable cell adhesion properties [6]. Electrospun fibrous gelatin and zein scaffolds were evaluated for cytocompatibility, stiffness and hydrolytic stability. The osteogenic differentiation of MC3T3-E1 pre-osteoblast cell line was evaluated on zein scaffolds. MC3T3-E1 cells were cultured in both standard growth media and induction media and evaluated for presence of osteogenic specific markers.

The materials and the methods of the present disclosure used in some embodiments will be described below. While the embodiments discuss the use of specific compounds and materials, it is understood that the present disclosure could employ other suitable materials. Similar quantities or measurements may be substituted without altering the methods embodied below.

Fabrication of Scaffolds: Fabrication of 24% (w/w) gelatin (bovine type B) in 60/40 acetic acid/water solutions and 30% (w/w) zein in 80/20 ethanol/water solutions were prepared for electrospinning. Solutions were electrospun using standard conditions and crosslinked. Cell Culture: Human mesenchymal stem cells (hMSCs), passage 5, were seeded at 32,000 cells/cm² onto gelatin and zein scaffolds, cut into discs and cultured in complete growth medium (DMEM, 10% FBS, 1% antibiotic/antimycotic) for up to 11 days. MC3T3-E1, passage 5, were cultured up to 21 days on zein scaffolds at 30,000 cells/cm² in either complete growth medium (GM; DMEM, 10% fetal bovine serum, 1% antibiotic/antimycotic) or osteogenic induction medium (OM; GM supplemented with 0.05 mM of ascorbic acid, 10 mM of β-glycerophosphate, and 100 nM of dexamethasone). Cell Growth: Quanti-iT PicoGreen® dsDNA reagent was utilized to quantify cell number at days 1, 7 and 11. Confocal imaging of hMSCs seeded on scaffolds was conducted at day 1 or day 7 and stained with Phalloidin (actin).

Osteogenic Differentiation Studies: Cell morphology and secretion of matrix proteins associated with osteogenic differentiation (osteocalcin) were evaluated with confocal imaging by immunostaining (anti-osteocalcin), rhodamine phalloidin (cytoskeletal actin filaments) and DAPI blue (cell nuclei stain). Matrix mineralization was quantified by the calcium assay kit following the manufacturer's protocol (BioAssay Systems) and evaluated by SEM with energy dispersive X-ray spectroscopy (EDX).

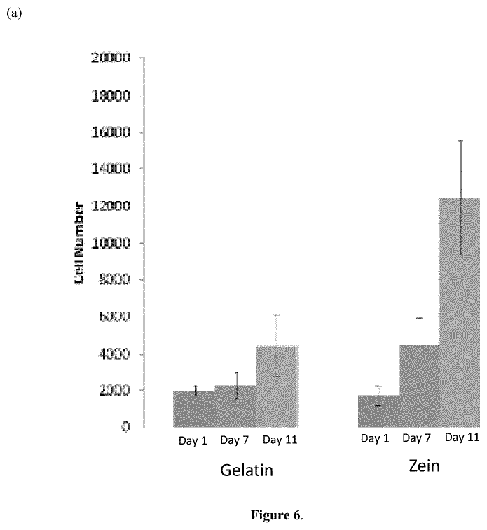

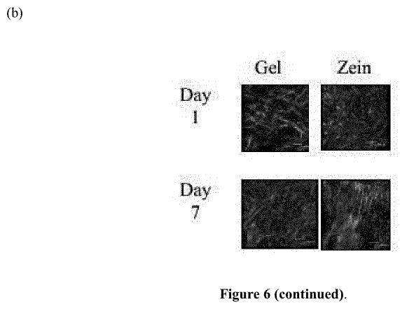

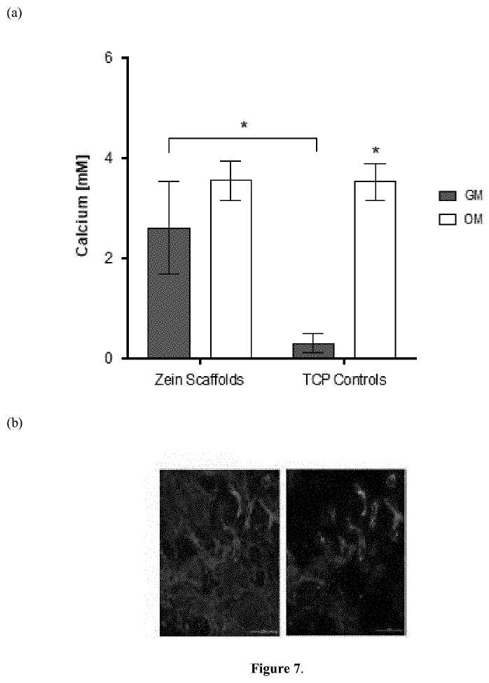

The results of the zein study will be described below. Referring to FIG. 6, cell number increased on zein scaffolds at later time points as compared to gelatin. With reference to FIG. 7, confocal images demonstrate viable cell attachment and standard hMSC morphology on both gelatin and zein scaffolds. Mechanical properties of the two scaffolds were evaluated by conducting tensile testing. The elastic modulus was 195 kPa±47 kPa (zein) and 194 kPa±53 kPa (gelatin). Hydrolytic stability studies of these scaffolds also exhibited similar percent weight loss of 7.4%±3.6% (zein) and 6.7%±1.0% (gelatin) over 42 days suggesting stability in hydrolytic conditions.

Osteogenic potential of MC3T3-E1 cells was observed when seeded on zein scaffolds. Confocal imaging of MC3T3-E1 cells on zein scaffolds in OM conditions expressed osteocalcin by day 21. Mineral deposits by the MC3T3-E1 cells were observed using SEM at day 21 on zein scaffolds. Calcium deposition was significantly higher for cells seeded on zein scaffolds than on TCP control in GM conditions suggesting osteogenic potential.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

### REFERENCES

[1] Gama, C. L. et al, Nat Chem Biol, 2006.
[2] Huang, G. P. et al, J Tissue Eng Regen Med, 2017.
[3] Huang, G. P. et al, Tissue Eng Part A, 2017.
[4] Jiang Q. Acta biomaterialia. 2010; 6(10):4042-51.
[5] Shukla R. Industrial crops and products. 2001; 13(3):171-92.
[6] Hoque M E. Polymers Research Journal. 2015; 9(1):15.

1. An implant for promoting bone and/or cartilage formation, said implant comprising a plurality of scaffolds arranged in a multi-layer stacked configuration;
wherein each scaffold comprises a mesh of polymer fibers; and
wherein the polymer fibers comprise gelatin, a plant-derived protein or a combination thereof.

2. The implant of claim 1, wherein the polymer fibers in at least one scaffold further comprise a sulfated polymer.

3. The implant of claim 2, wherein said sulfated polymer is selected from the group consisting of cellulose sulfate, starch sulfate and chitin sulfate.

4. The implant of claim 3, wherein said sulfated polymer is cellulose sulfate.

5. The implant of claim 4, wherein said cellulose sulfate is a fully sulfated cellulose sulfate (fSC), partially sulfated cellulose sulfate (pSC) or a combination thereof.

6. The implant of claim 1, wherein the polymer fibers are electrospun.

7. The implant of claim 1, wherein the polymer fibers comprise gelatin.

8. The implant of claim 7, wherein the polymer fibers are crosslinked.

9. The implant of claim 8, wherein the polymer fibers are crosslinked with a crosslinker selected from the group consisting of N-(3-dimethyl aminopropyl)-N′-ethyl carbodiimide with N-hydroxysuccinimide (EDC/NHS), genipen and a combination thereof.

10. The implant of claim 1, wherein the polymer fibers comprise a plant-derived protein.

11. The implant of claim 10, wherein the plant-derived protein is selected from the group consisting of zein protein and soy protein.

12. The implant of claim 11, wherein the plant-derived protein is zein protein.

13. The implant of claim 10, wherein the polymer fibers are crosslinked.

14. The implant of claim 13, wherein the polymer fibers are crosslinked with an epoxy-based crosslinker.

15. The implant of claim 14, wherein said epoxy-based crosslinker is trimethylolpropane triglycidyl ether (TMPGE).

16. The implant of claim 1, wherein in at least one scaffold
the polymer fibers have an average fiber diameter of between about 100 nm and about 100 μm; or
the mesh of polymer fibers exhibits interfiber spacing of between about 10 μm and about 200 μm.

17. (canceled)

18. The implant of claim 1, wherein the implant is hydrolytically stable.

19-22. (canceled)

23. A method for repairing a cartilage and/or an osteochondral defect in a subject in need thereof, said method comprising disposing in the cartilage and/or the osteochondral defect the implant of claim 1.

24. A method for repairing a cartilage and/or an osteochondral defect in a subject in need thereof, said method comprising disposing in the cartilage and/or the osteochondral defect a plurality of scaffolds arranged in a multi-layer stacked configuration;
wherein each scaffold comprises a mesh of polymer fibers; and
wherein the mesh of polymer fibers comprises gelatin, a plant-derived protein or a combination thereof.

25-44. (canceled)

45. A scaffold for promoting bone and/or cartilage formation, said scaffold comprising a mesh of polymer fibers;
wherein the polymer fibers comprise a plant-derived protein and a sulfated polymer.

46-53. (canceled)
